(19) United States
(12) Patent Application Publication
(10) Pub. No.: US 2025/0361474 A1
(43) Pub. Date: Nov. 27, 2025

(54) BIONIC ORGAN DEVICE

(71) Applicant: DARWIN PRECISIONS CORPORATION, Hsinchu County (TW)

(72) Inventors: Ting-Chieh Yang, Hsinchu County (TW); Chung-Yi Huang, Hsinchu County (TW)

(21) Appl. No.: 19/216,733

(22) Filed: May 23, 2025

(30) Foreign Application Priority Data
May 24, 2024 (TW) 113119392

(51) Int. Cl. C12M 1/42; C12M 1/00; C12M 1/12

(52) U.S. Cl. CPC C12M 35/04; C12M 23/26; C12M 25/02

(57) ABSTRACT

A bionic organ device includes an organ chip and a magnetic field generating module. The organ chip includes a first body, a second body, a porous membrane, and a magnetically-driven flexible body. The porous membrane is disposed between the first body and the second body and forms a channel system with the first body and the second body. The at least one magnetically-driven flexible body is disposed in the first body, the second body, or a combination thereof, and is adjacent to the channel system. The magnetic field generating module is disposed outside the organ chip and is adapted to generate a magnetic field. The magnetic field passes through the at least one magnetically-driven flexible body and causes the at least one magnetically-driven flexible body to deform in response to a magnetic force in the magnetic field.

### FIELD OF THE INVENTION

The present invention relates to a bionic technology, specifically to a bionic organ device that can simulate the microenvironment in an organism.

### BACKGROUND OF THE INVENTION

Traditional cell culture models cannot reflect the complicated physiological functions of tissues and organs in an organism. Animal experiments have drawbacks such as long period and high cost. Organ chips reconstruct the in vivo physiological environment of organs, simulating the structures, microenvironment, and physiological functions of organs in the organism. Organ chips also offer accurate parameter control, along with advantages of miniaturization, integration, high efficiency, and reduced cost. To simulate the dynamic microenvironment in the organism or the stretching and contraction of cells, current organ chips are equipped with the vacuum systems that achieve bionic effects through vacuum application. However, the vacuum system which carries out the stretching also pulls on the membrane to which cells are attached, potentially causing membrane damage and malfunction of the organ chips. In addition, the manufacturing of the vacuum system is complicated and improvement is therefore required.

### SUMMARY OF THE INVENTION

The invention provides a bionic organ device which can be used to simulate dynamic microenvironment of organs, and has a more simplified structure which is beneficial to having a streamlined manufacturing process, reducing costs, and improving yields.

The bionic organ device provided by the invention comprises an organ chip and a magnetic field generating module. The organ chip comprises a first body, a second body, a porous membrane, and at least one magnetically-driven flexible body. The porous membrane is disposed between the first body and the second body and forms a channel system with the first body and the second body. The at least one magnetically-driven flexible body is disposed in the first body, the second body, or a combination thereof, and is adjacent to the channel system. The magnetic field generating module is disposed outside the organ chip and is adapted to generate a magnetic field, and the magnetic field passes through the at least one magnetically-driven flexible body and causes the at least one magnetically-driven flexible body to deform in response to a magnetic force in the magnetic field.

The present invention utilizes the magnetically-driven flexible body and the magnetic field generating module in combination with the porous membrane, enabling the simulation of the dynamic microenvironment in the organism and the performances of organs, tissues, or cells in such dynamic microenvironment, and making it more convenient to use. Further, the organ chip of the present invention has a simplified structure, which is beneficial to having a more streamlined manufacturing process, reducing costs, and improving yields.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIG. 1 is a schematic three-dimensional view of a bionic organ device according to an embodiment of the invention;

FIG. 2 is a partial schematic three-dimensional exploded view of a bionic organ device according to an embodiment of the invention;

FIG. 3 is a schematic cross-sectional view taken along line A-A″ in FIG. 1;

FIG. 4 is a schematic cross-sectional view of a magnetically-driven flexible body according to an embodiment of the invention;

FIG. 5 is a schematic view of an operation of a bionic organ device according to an embodiment of the invention; and

FIG. 6 is a schematic view of an operation of a bionic organ device accordingly to another embodiment of the invention.

### DETAILED DESCRIPTION OF PREFERRED EMBODIMENTS

The foregoing and other technical contents and other features and advantages of the present invention will be clearly presented from the following detailed description of a preferred embodiment in cooperation with the accompanying drawings. Directional terms mentioned in the following examples are only used to describe directions referring to the attached drawings. Therefore, the directional terms used are for illustration and not for limitation. In addition, terms such as “first” and “second” involved in the description or claims are merely used for naming the elements or distinguishing different embodiments or ranges rather than limiting the upper limit or lower limit of the quantity of the elements.

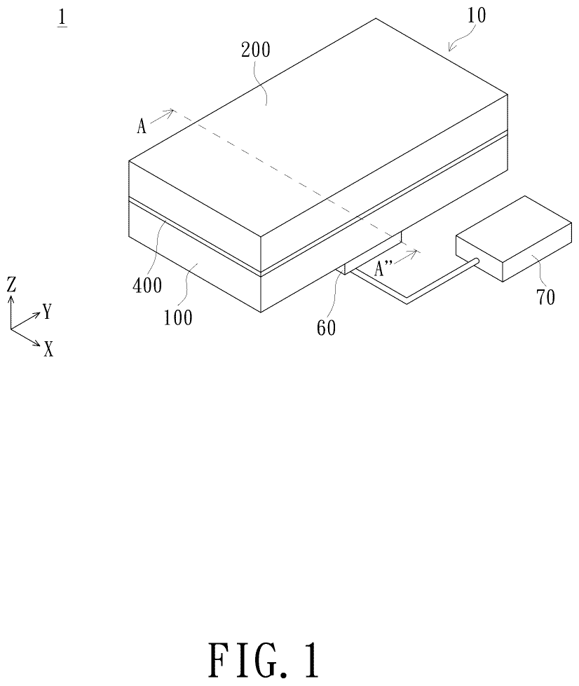

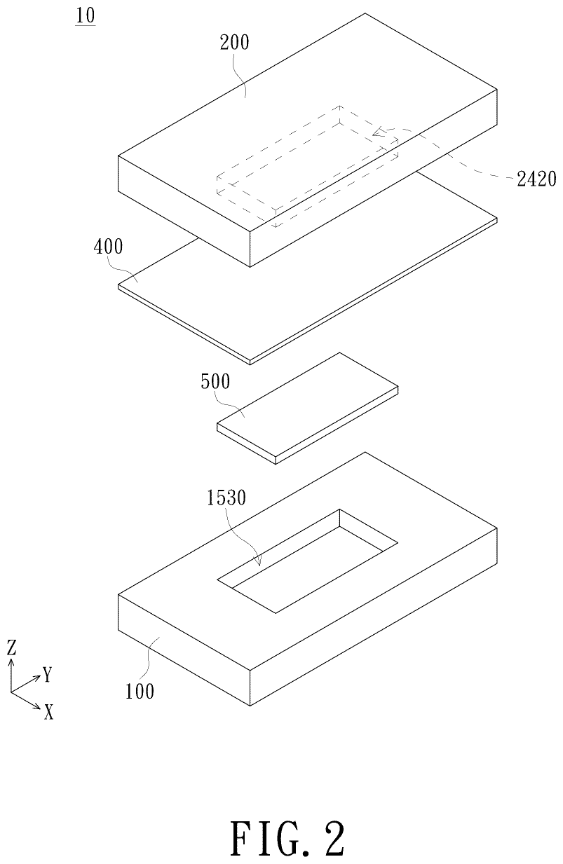

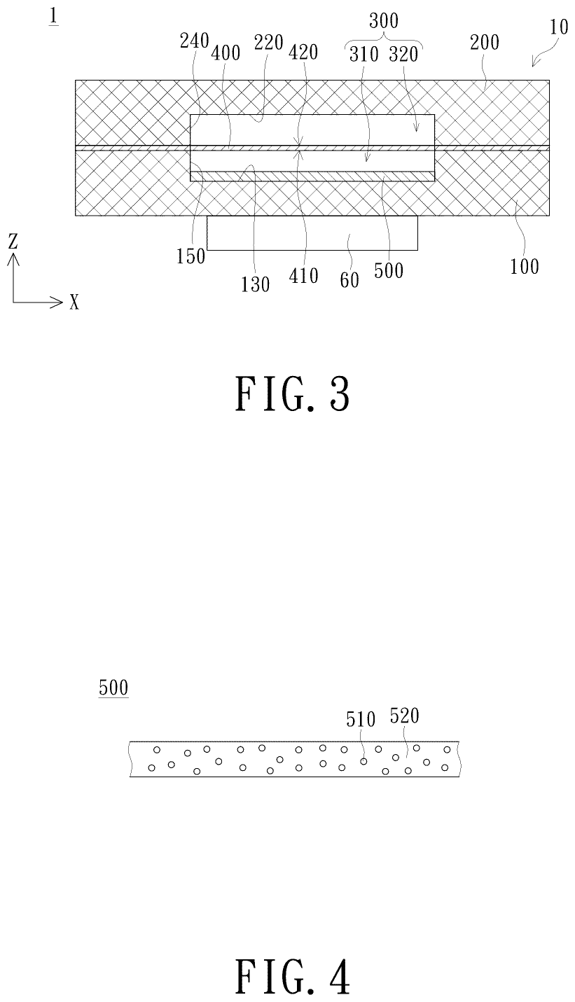

FIG. 1 is a schematic three-dimensional view of a bionic organ device according to an embodiment of the invention, FIG. 2 is an exploded view of FIG. 1, and FIG. 3 is a schematic cross-sectional view taken along line A-A″ in FIG. 1. As shown in FIGS. 1 to 3, the bionic organ device of the embodiment of the present invention includes an organ chip 10 and a magnetic field generating module 60. The magnetic field generating module 60 is disposed outside the organ chip 10 and is adapted to generate a magnetic field (described in detail later), allowing the organ chip 10 to simulate, for example, the dynamic microenvironment of organs, tissues, or cells in an organism. The magnetic field generating module 60 can be disposed close to the organ chip 10, for example, on the organ chip 10, but is not limited thereto. The relative position between the magnetic field generating module 60 and the organ chip 10 is that, in principle, the organ chip 10 is within the range of the influence of the magnetic field.

The organ chip 10 includes a first body 100, a second body 200, a porous membrane 400, and at least one magnetically-driven flexible body 500. The porous membrane 400 is disposed between the first body 100 and the second body 200 and forms a channel system 300 with the first body 100 and the second body 200. FIGS. 1 to 3 illustrate primarily the positional relationship among the first body 100, the second body 200, the channel system 300, the porous membrane 400, and the magnetically-driven flexible body 500. It is known that the relative sizes of these five components do not necessarily have to be as shown in the figures. The magnetically-driven flexible body 500 is disposed in the first body 100, the second body 200, or a combination thereof. Further, the magnetically-driven flexible body 500 is within the influence range of the magnetic field and can affect the channel system 300 and the porous membrane 400 in response to the magnetic force.

As shown in FIGS. 2 and 3, the first body 100 and the second body 200 each may have a groove-like structure with an accommodating space and an opening. In the embodiment of the present invention, the first body 100 may include a bottom portion 130 and a wall portion 150. The wall portion 150 is formed along a perimeter of the bottom portion 130, and the bottom portion 130 and the wall portion 150 together form an accommodating space 1530 (i.e., a first accommodating space 1530) within the first body 100. The structure of the second body 200 may be the same as or similar to that of the first body 100. In the embodiment of the present invention, the second body 200 may include a bottom portion 220 and a wall portion 240 formed along a perimeter of the bottom portion 220, and the bottom portion 220 and the wall portion 240 together form an accommodating space 2420 (i.e., a second accommodating space 2420) within the second body 200. The magnetically-driven flexible body 500 can be further disposed in the accommodating space 1530 of the first body 100 or the accommodating space 2420 of the second body 200. Alternatively, the at least one magnetically-driven flexible body 500 is disposed in both the accommodating spaces 1530 and 2420.

In the embodiment of the present invention, the first body 100 and the second body 200 are combined with each other through arranging the respective accommodating spaces 1530 and 2420 to face each other. The porous membrane 400 can be connected to a surface of the first body 100 facing the second body 200 and a surface of the second body 200 facing the first body 100, and therefore be opposite respectively to the bottom portion 130 of the first body 100 and the bottom portion 220 of the second body 200. The connection among the first body 100, the second body 200, and the porous membrane 400 can be achieved by means of one or more known methods such as thermal pressing, welding, adhesive bonding, or other means capable of achieving the connection, depending on the materials of the first body 100, the second body 200, and the porous membrane 400. The materials of the first body 100 and the second body 200 can be plastics such as but not limited to polycarbonate (PC), polyethylene terephthalate (PET), polypropylene (PP), polyvinyl chloride (PVC), and polydimethylsiloxane (PDMS). The porous membrane 400 can be made from artificially synthesized polymer material such as polyethylene terephthalate (PETE), polydimethylsiloxane (PDMS), polyurethane, styrene-ethylene-butylene-styrene (SEBS), poly (hydroxyethyl methacrylate) (pHEMA), polyethylene glycol, polyvinyl alcohol, or polycarbonate (PC), but is not limited thereto.

As described above, the porous membrane 400, the first body 100, and the second body 200 form the channel system 300. Specifically, the porous membrane 400 forms a first channel 310 of the channel system 300 with the first body 100, and the porous membrane 400 forms a second channel 320 of the channel system 300 with the second body 200. The first channel 310 and/or the second channel 320 is able to allow at least one fluid (not shown) to pass through or remain within. Additionally, the porous membrane 400 has a first membrane surface 410 and a second membrane surface 420 with the first membrane surface 410 located in the first channel 310 and the second membrane surface 420 located in the second channel 320. The porous membrane 400 is adapted to be as a cell-attaching membrane. In the embodiment of the present invention, the porous membrane 400 can have a pore size on the nanometer (nm) scale, such as dozens of nanometers, tens of nanometers, or hundreds of nanometers. The porous membrane 400 preferably has compliance and is stretchable and extendable. When the porous membrane 400 is used as a cell-attaching membrane, the first membrane surface 410 and the second membrane surface 420 can be for cell attachment, and fluids in the first channel 310 and/or the second channel 320 can come into contact with the cells on the first membrane surface 410 and/or the second membrane surface 420. Due to the porosity of the porous membrane 400, small molecules are able to pass through the porous membrane 400 and move between the first channel 310 and the second channel 320. This allows the organ chip 10 to simulate the phenomena of organs, tissues, or cells in the organism under dynamic microenvironment. The channel system 300 may further include input/output ports (not shown), which can be arranged on the organ chip 10 in any known manner for the intercommunication between the interior and exterior of the organ chip 10.

As described above, the at least one magnetically-driven flexible body 500 can be further disposed in the accommodating space 1530, the accommodating space 2420, or a combination thereof. In a preferred embodiment of the present invention, the magnetically-driven flexible body 500 can be further disposed on the bottom portion 130 of the first body 100 and preferably is opposite directly to the porous membrane 400. The magnetically-driven flexible body 500 and the porous membrane 400 can be separated by the first channel 310. When the magnetically-driven flexible body 500 responds to the magnetic force generated by the magnetic field generating module 60, the magnetically-driven flexible body 500 can deform, affect the channel system 300, and influence the porous membrane 400 through, for example, the first channel 310.

FIG. 4 is a schematic cross-sectional view of a magnetically-driven flexible body according to an embodiment of the present invention. As shown in FIG. 4, the magnetically-driven flexible body 500 can include a magnetic material 510 and a flexible sheet 520. The magnetic material 510 is further a strong magnetic material, including ferromagnetic and ceramic ferromagnetic materials, such as but not limited to iron, cobalt, nickel, alloys thereof, or compounds thereof. In addition, the magnetic material 510 may vary in properties such as saturation magnetization and dispersibility, where high saturation magnetization indicates a stronger response to magnetic force, and high dispersibility helps prevent aggregation. In the embodiment of the present invention, the magnetic material 510 is preferably a ferromagnetic material or a ceramic ferromagnetic material, such as iron, cobalt, nickel, alloys thereof, or compounds thereof, with appropriate saturation magnetization and dispersibility, and is distributed in particle form within the flexible sheet 520. FIG. 4 illustrates mainly the positional relationship between the particles of the magnetic material 510 and the flexible sheet 520. It is known that the relative sizes of these two do not necessarily have to be as shown in the figure. The particles of the magnetic material 510 can have a size measured in micrometers (μm), such as several micrometers, dozens of micrometers, or tens of micrometers.

The flexible sheet 520 can be stretchable and expandable, endowing the magnetically-driven flexible body 500 with deformability. A material of the flexible sheet 520 can be either natural or synthetic polymer material. In some embodiments of the present invention, the material of the flexible sheet 520 includes hydrogel. The hydrogel, for example, can be obtained by crosslinking of acrylic acid or its derivatives, acrylamide or its derivatives, and/or hydroxyethyl methacrylate or its derivatives, but is not limited thereto. When using the hydrogel as the material for the flexible sheet 520, for example, a powder of the magnetic material 510 can be added to the hydrogel in a sol or fluid state and thoroughly mixed, and then the mixture is formed into the magnetically-driven flexible body 500 that includes the flexible sheet 520 and the magnetic material 510. A mixing ratio of the magnetic material 510 to the hydrogel can be, for example, in a weight ratio of 0.5:1 to 4:1, such as 1:1, 1.5:1, 2:1, 2.5:1, 3:1, or 3.5:1. The forming method may involve changes in temperature, such as heating or cooling, but is not limited thereto.

In a preferred embodiment of the invention, the magnetic material 510 for being added to and mixed with the hydrogel includes a carbonyl magnetic iron powder. The carbonyl magnetic iron powder can have an appropriate size, for example, having a particle diameter of 5 to 9 μm. The magnetically-driven flexible body 500 can be pre-made and then disposed on the bottom portion 130, or can be directly fabricated and molded within the accommodating space 1530. For example, a hydrogel in the sol or fluid state mixed with the magnetic material 510 can be injected into the accommodating space 1530, and the disposition of the magnetically-driven flexible body 500 on the bottom portion 130 is completed after the hydrogel is modeled. In a preferred embodiment of the present invention, the magnetically-driven flexible body 500 can be further fastened to the bottom portion 130.

As described above, the magnetically-driven flexible body 500 has deformability and can respond to the magnetic force generated by the magnetic field generating module 60. Further, the magnetic force is adapted to attract or repel the magnetic material 510 of the magnetically-driven flexible body 500, and the magnetic attraction or repulsion further causes the magnetically-driven flexible body 500 to deform due to the compliance of the flexible sheet 520. For example, when the direction of the magnetic attraction or repulsion is toward the channel system 300 or the porous membrane 400, the magnetically-driven flexible body 500 may bulge or protrude in a direction toward the channel system 300 or the porous membrane 400. In addition, for example, when there is a higher content of the magnetic material 510 or a higher compliance of the flexible sheet 520, the same magnetic attraction or magnetic repulsion may cause a larger amount of deformation. In the preferred embodiment of the present invention, the bulging or protrusion of the magnetically-driven flexible body 500 toward the channel system 300 may also result in compression on the channel system 300. For example, when the magnetically-driven flexible body 500 compresses the first channel 310, the pressure in the first channel 310 increases, thereby being able to push the porous membrane 400, and the porous membrane 400 can therefore stretch due to its compliance.

In the embodiments of the present invention, the magnetic field generating module 60 is preferably powered by electricity to generate the magnetic field. As shown in FIG. 1, the bionic organ device 1 can further include a power supply 70, which is electrically connected to the magnetic field generating module 60 and supplies power to the magnetic field generating module 60. Preferably, the magnetic field generating module 60 can include an electromagnet. The power supply 70 supplies electric current, causing the electromagnet in the magnetic field generating module 60 to have a magnetic effect and generate a magnetic field. The magnetic field generating module 60 may further include an electromagnet capable of generating a magnetic force of 0 to 30 Kg and a driving voltage of 12 V to 110 V. In some embodiments of the present invention, the magnetic field generating module 60 may be, for example, an electromagnet that is able to generate a magnetic force of 10 Kg and a driving voltage of, for example, 12 V.

As described above, the organ chip 10 is within the influence range of the magnetic field generated by the magnetic field generating module 60, and the magnetic field generating module 60 is disposed on the organ chip 10. In some embodiments of the present invention, as shown in FIGS. 1 to 3, the magnetic field generating module 60 is disposed on the first body 100 and is close to the magnetically-driven flexible body 500, for example, being separated from the magnetically-driven flexible body 500 by the bottom portion 130 of the first body 100. The specific location of the magnetic field generating module 60 on the first body 100 can be further determined based on factors such as magnetic field direction, magnetic field strength, magnetic force magnitude, and magnetic flux density.

In a preferred embodiment of the present invention, the bionic organ device 1 may further include a control module (not shown). The control module may include components such as a microprocessor, a microcontroller, or a microcomputer and is electrically connected to the magnetic field generating module 60, the power supply 70, or a combination thereof. This electrical connection can be achieved through various known means and may include configurations such as circuit boards, adapters, etc., but is not limited thereto. The control module may be loaded with a control program that outputs a control signal to turn the power supply 70 and the magnetic field generating module 60 on or off and to generate the magnetic field. The control module can further control the magnetic field strength, the magnetic force magnitude, the magnetic flux density, or a combination thereof. In some embodiments of the present invention, the control module may also control the direction of the magnetic field.

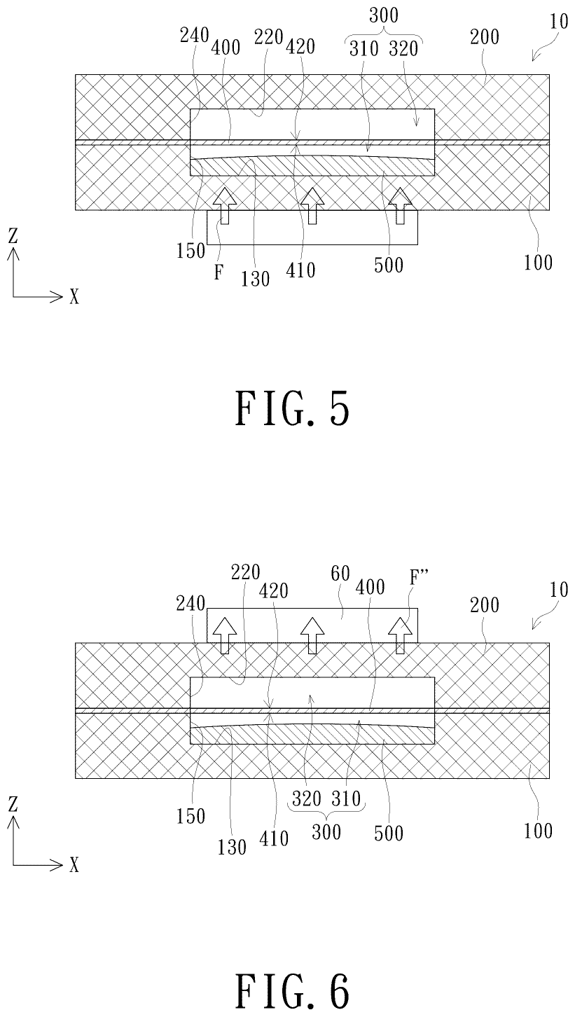

An operation of the bionic organ device 1 is illustrated with the embodiments shown in FIGS. 1 to 3 and FIG. 5 as an example. As shown in FIG. 5, in some embodiments of the present invention, when the power supply 70 supplies power to the magnetic field generating module 60, the generated magnetic field preferably can exert a magnetic repulsion force F on the magnetically-driven flexible body 500 and drive the magnetically-driven flexible body 500 to bulge in the direction toward the porous membrane 400, thereby compressing the first channel 310, making the pressure in the first channel 310 to increase, and pushing the porous membrane 400 to stretch (not shown). Moreover, the porous membrane 400 may stretch periodically. The periodic stretching of the porous membrane 400 can be achieved, for example, by periodically supplying power by the power supply 70 to generate the magnetic repulsion force F periodically on the magnetically-driven flexible body 500. When the porous membrane 400 stretches periodically, for example, the cells attaching thereto may stretch periodically.

In some embodiments of the present invention, when the pressure in the first channel 310 increases as described above, a pressure difference between the first channel 310 and the second channel 320 can be created. The pressure difference may be used to drive the movement of the fluids and substances in the channel system 300 and between two sides of the porous membrane 400. On the other hand, in some embodiments, when the pressure in the first channel 310 increases and the porous membrane 400 stretches, the porous membrane 400 may have an effect of letting cells stretch. As a result, the organ chip 10 is able to simulate the performances of organs, tissues, and cells in the organism under dynamic microenvironment.

The magnetic field generating module 60 is not limited to being disposed close to the magnetically-driven flexible body 500. For example, the magnetic field generating module 60 and the magnetically-driven flexible body 500 can be separately disposed on the first body 100 and the second body 200. In some embodiments of the present invention, as shown in FIG. 6, the magnetically-driven flexible body 500 is disposed in the accommodating space 1530 of the first body 100, and the magnetic field generating module 60 is disposed on the second body 200. In this case, when the power supply 70 supplies power to the magnetic field generating module 60, the generated magnetic field preferably exerts a magnetic attraction force F″ on the magnetically-driven flexible body 500, driving it to bulge towards the porous membrane 400 and to compress the first channel 310, and causing the porous membrane 400 to stretch.

In addition to causing deformation of the magnetically-driven flexible body 500, in some embodiments of the present invention, the magnetic repulsion force F or the magnetic attraction force F″ may cause displacement of the magnetically-driven flexible body 500. For example, the magnetically-driven flexible body 500 may move toward the first channel 310, compress the first channel 310, and increase the pressure therein, thereby pushing the porous membrane 400 to stretch. The amount of deformation or displacement of the magnetically-driven flexible body 500 may vary depending on the magnitude of the magnetic repulsion force F or the magnetic attraction force F″. For example, when the magnetic repulsion force F or the magnetic attraction force F″ is greater, the amount of deformation or displacement is also greater. In some embodiments of the present invention, the magnetic repulsion force F or the magnetic attraction force F″ can be, for example, in a range of 0 to 100 mT, and preferably less than or equal to 70 mT. The amount of deformation or displacement of the magnetically-driven flexible body 500 can further be determined by the extent to which the porous membrane 400 can stretch and the size of the channel system 300. In the embodiment of the present invention, the amount of deformation or displacement is preferably not more than 203.5 μm and at least 1 μm, such as 1 μm, 25 μm, 50 μm, 75 μm, 100 μm, 125 μm, 150 μm, 175 μm, or 200 μm. The deformation amount can be, for example, a distance between an apex of the bulging magnetically-driven flexible body 500 and its original position.

In summary, the present invention uses the magnetically-driven flexible body 500 and the magnetic field generating module 60 to provide a means that is entirely different from traditional vacuum approaches and is able to simulate the dynamic microenvironment in the organism and the performances of organs, tissues, or cells in that dynamic microenvironment. Users can set up the power supply 70 and/or the control module from the outside of the organ chip 10 to achieve the effect of simulating the dynamic microenvironment and the performances of organs, tissues, or cells in the dynamic microenvironment, which is more convenient compared to traditional methods that rely on vacuum systems for gas extraction and delivery. In addition, unlike traditional vacuum systems that require channels for gas extraction and delivery to be arranged inside the organ chip, the organ chip 10 of the present invention can have a simplified structure and a streamlined process thereof due to no requirement for the vacuum system, which is beneficial for reducing costs and improving yields.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A bionic organ device, comprising an organ chip and a magnetic field generating module, the organ chip comprising a first body, a second body, a porous membrane, and at least one magnetically-driven flexible body, wherein:
the porous membrane is disposed between the first body and the second body and forms a channel system with the first body and the second body;
the at least one magnetically-driven flexible body is disposed in the first body, the second body, or a combination thereof and is adjacent to the channel system; and
the magnetic field generating module is disposed outside the organ chip and is adapted to generate a magnetic field, and the magnetic field passes through the at least one magnetically-driven flexible body and causes the at least one magnetically-driven flexible body to deform in response to a magnetic force in the magnetic field.

2. The bionic organ device according to claim 1, further comprising a power supply, wherein the power supply is electrically connected to the magnetic field generating module and is adapted to supply power to the magnetic field generating module.

3. The bionic organ device according to claim 2, wherein the magnetic field generating module comprises an electromagnet, the power supply further supplies an electric current, and the electric current is adapted to cause the electromagnet to have a magnetic effect and to generate the magnetic field.

4. The bionic organ device according to claim 1, wherein the at least one magnetically-driven flexible body comprises a magnetic material and a flexible sheet.

5. The bionic organ device according to claim 4, wherein the magnetic material comprises a plurality of particles of iron, cobalt, nickel, alloys thereof, or compounds thereof, and the plurality of particles of the magnetic material are distributed in the flexible sheet.

6. The bionic organ device according to claim 4, wherein the magnetic force in the magnetic field is adapted to attract the magnetic material to drive the at least one magnetically-driven flexible body to deform.

7. The bionic organ device according to claim 4, wherein the magnetic force in the magnetic field is adapted to repel the magnetic material to drive the at least one magnetically-driven flexible body to deform.

8. The bionic organ device according to claim 4, wherein the magnetic material is a carbonyl magnetic iron powder, the flexible sheet is made from hydrogel, and a weight ratio of the carbonyl magnetic iron powder to the hydrogel is 0.5:1 to 4:1.

9. The bionic organ device according to claim 1, wherein the first body comprises a first bottom portion and a first wall portion, the first wall portion is formed along a perimeter of the first bottom portion, the first bottom portion and the first wall portion form a first accommodating space in the first body, and the at least one magnetically-driven flexible body is further disposed in the first accommodating space and is located in the first bottom portion.

10. The bionic organ device according to claim 9, wherein the magnetic field generating module is further disposed on the first body.

11. The bionic organ device according to claim 9, wherein the second body comprises a second bottom portion and a second wall portion, the second wall portion is formed along a perimeter of the second bottom portion, the second bottom portion and the second wall portion form a second accommodating space in the second body; the second body is combined with the first body through arranging the second accommodating space to face the first accommodating space, and the porous membrane is disposed between the first body and the second body and is spaced apart from and opposite to the at least one magnetically-driven flexible body.

12. The bionic organ device according to claim 10, wherein the magnetic field generating module is separated from the at least one magnetically-driven flexible body by the first bottom portion.

13. The bionic organ device according to claim 1, wherein a deformation of the at least one magnetically-driven flexible body comprises a bulging toward the porous membrane.

14. The bionic organ device according to claim 13, wherein the channel system comprises a first channel and a second channel, the first channel is located between the first body and the porous membrane, the second channel is located between the second body and the porous membrane, and the bulging of the at least one magnetically-driven flexible body toward the porous membrane is adapted to compress the first channel.

15. The bionic organ device according to claim 14, wherein the at least one magnetically-driven flexible body bulges toward the porous membrane, compresses the first channel, and increases a pressure in the first channel.

16. The bionic organ device according to claim 13, wherein the bulging of the at least one magnetically-driven flexible body toward the porous membrane is adapted to push the porous membrane to stretch.

17. The bionic organ device according to claim 9, wherein the magnetic field generating module is further disposed on the second body.
